(19) United States
(12) Patent Application Publication
(10) Pub. No.: US 2009/0069715 A1
(43) Pub. Date: Mar. 12, 2009

(54) Urine collection device

(76) Inventor: Judith Holloway, New Hope, PA (US)

Correspondence Address: JAMES RAY & ASSOCIATES, 3640 PITCAIRN ROAD, MONROEVILLE, PA, 15146, US

(21) Appl. No.: 12/283,445

(22) Filed: Sep. 12, 2008

Related U.S. Application Data
(60) Provisional application No. 60/971,592, filed on Sep. 12, 2007.

(51) Int. Cl. A61B 5/20

(52) U.S. Cl. 600/581

(57) ABSTRACT

A stent urine collection device is used to collect urine that would otherwise leak from the stent. The collection device includes a pair openings in the collection bag, an inverted open channel to receive the stents where the stents are securely retained in the channel and where the channel has a drainage means to allow the urine to collect in the bag.

### CROSS REFERENCE TO RELATED APPLICATION

This patent application is related to and claims priority from U.S. Provisional Patent Application Ser. No. 60/971,592 filed Sep. 12, 2007.

### FIELD OF THE INVENTION

The present invention relates, in general, to urine collection and, more particularly, this invention relates to with urine collection device for use with a catheter.

### BACKGROUND OF THE INVENTION

Prior to the conception and development of the present invention, as is generally well known in the prior art, to provide for drainage for a lighted stent for use with a catheter. The stent surrounding the fiber optic element is typically inserted in a female urethra laproscopically or otherwise and causes urine to leak outside of a Foley catheter collection system. The Foley catheter is a tube inserted in the bladder to continuously drain urine. The problem of urine leakage has been addressed by U.S. Pat. No. 4,248,214 issued to Hannah et al. provides for a drainage adapter for the catheter. However, the drainage is limited to and proximate to the lighted catheter insertion device. The device in Hannah does not address continuing leakage or storage away from the doctor. U.S. Pat. No. 5,531,741 issued to Barbacci has apertures in the stent to allow drainage from sheathing surrounding the fiber optic thread, but without a collection system.

### SUMMARY OF THE INVENTION

The present invention provides a collection device for urine that would other wise leak on to a stent used in laproscopic surgery or diagnostic procedures. Typically a foley catheter is inserted in a patients bladder to continuously drain the bladder. However, use of a lighted stent causes leakage from the patient's bladder. The present invention collects the leakage. A collection bag is provided with openings or flaps on lateral sides of the bag. The interior of the bag has an open channel preferably in the shape of an inverted arc. The channel has a drainage means, preferably apertures in the channel to allow the leaked urine to drain into the collection bag. The stent enters the bag through an opening and is received by the open channel. The stent exits through the other opening. The stent is retained by a retention means. Preferably the retention means is at least one paper tape attached to the lateral side of the collection bag and has a side coated with an adhesive to hold the stent in place.

The collection preferably has an aperture in the bottom of the bag with a fitting that accommodates a cap or foley catheter tube so that the collection bag, tube and another collection bag are in fluid communication.

The bag has measurement marks printed on the bag corresponding to the volume of the bag. When used with a cap, the patient's urine volume output can be accurately measured at the leaked urine is captured and measure in the collection bag.

The bag preferably has a securing means. Preferably the securing means is a pair a straps attached to the lateral slides of the collection bag to secure the bag against free movement or swinging. The bag can be secured to the patient's leg, a bed or other apparatus.

### OBJECTS OF THE INVENTION

It is, therefore, one of the primary objects of the present invention to provide collection device used with lighted stents.

Another object of the present invention is to provide a device that collects leaks in laproscopic surgery and diagnostic procedures.

Still another object of the present invention is to provide a device that will allow for accurate urine output measurements accounting for urine leakage.

Yet another object of the present invention is to provide a device that optionally collects urine and is in fluid communication with further drainage tube.

An additional object of the present invention is to provide a device to collect leakage and secure the stent to a collection bag.

A further object of the present invention is providing a collection bag for leakage that is secured to the patient or bed to prevent free movement.

In addition to the various objects and advantages of the present invention described with some degree of specificity above it should be obvious that additional objects and advantages of the present invention will become more readily apparent to those persons who are skilled in the relevant art from the following more detailed description of the invention, particularly, when such description is taken in conjunction with the attached drawing figures and with the appended claims.

### BRIEF DESCRIPTION OF THE DRAWINGS

The single FIGURE is a cross section view of the device.

### BRIEF DESCRIPTION OF A PRESENTLY PREFERRED AND VARIOUS ALTERNATIVE EMBODIMENTS OF THE INVENTION

Prior to proceeding to the more detailed description of the present invention it should be noted that, for the sake of clarity and understanding, identical components that have identical functions have been identified with identical reference numerals throughout the several views illustrated in the drawing figures.

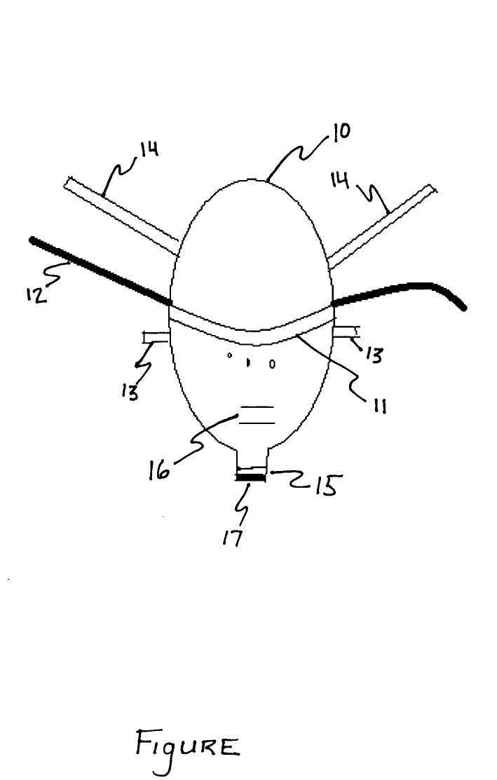

Reference is now made, more particularly, to FIG. 1 a urine collection bag 10. The collection bag 10 is manufactured of a fluid impervious material. Preferably, the material is plastic. The collection bag 10 includes openings on opposing lateral sides of the bag (not shown) the openings with a predetermined shape and size. The openings are preferably slots, but circular or oval openings could be used. Within the bag is an inverted arc open channel 11 with the open on the top of the channel. At least one lighted stent 12 such as those that are used is laproscopic surgery and diagnostic procedures enters the bag through one of the openings is received by the channel 11 and exits the collection bag 10 through the opposing side opening. Preferably, the channel 11 and openings can accommodate two stents.

The channel 11 has a drainage means on at least a portion of its surface (not shown). Preferably the drainage means are slots or circular apertures through the channel 10. This allows urine what is has leaked from the bladder onto the stent 12 to drain into the collection bag 10. The drops of urine are shown draining from the stent 12 and channel 11.

The stent 12 is held into place by a stent retention means on at least one lateral side of the bag 10. Preferably, the stent is held in place by a pair of paper tapes 15 with one side of the tape 15 attached to the collection bag 10 and the inwardly facing surface coated with a pressure sensitive adhesive. Another embodiment of the retaining means is a snap closure (not shown) generally adjacent to at least one opening, preferably both openings, to hold the stent 12 or stents in place.

Preferably, the collection bag 10 has an aperture through the bottom of the bag 10 and having a fitting 15 that can receive a cap 17 or conventional foley catheter drainage tube (not shown). When used with a tube, the collection bag 10 would be in fluid communication with the tube and another collection bag (not shown).

The collection preferably has lines as measurement marks 16 printed on the collection bag 10. When the collection bag is used with a cap 17 this allows for the accurate measurement of urine volume output of a patient, but capturing the otherwise leaked urine.

A securing means is attached to the collection bag a predetermined location to secure the collection bag 10 against free movement or swinging. Preferably, straps 14 are attached to opposing sides of the collection bag 10. The straps 14 are manufactured preferably of cloth, paper or a hook and loop fastener. The collection bag can be secured to the patient's leg or to a bed or other apparatus.

While a presently preferred and various alternative embodiments of the present invention have been described in sufficient detail above to enable a person skilled in the relevant art to make and use the same it should be obvious that various other adaptations and modifications can be envisioned by those persons skilled in such art without departing from either the spirit of the invention or the scope of the appended claims.

I claim:

1. A urine collection device with a lighted stent and foley catheter comprising:
a fluid impervious collection bag of first predetermined material;
a pair of opposing openings with a first predetermined size and first predetermined shape;
an inverted arc shaped channel of a second predetermined size and shape receiving at least one of such lighted stents within the interior of said channel; the top of said channel being open;
a drainage means on at least a portion of said arc shaped channel within said collection bag; and
a stent retention means generally adjacent at least one of said openings.

2. A urine collection device of claim 1 wherein said drainage means are a plurality of aperture through said channel with a third predetermined shape.

3. A urine collection device of claim 2 wherein said third predetermined shape is one of slots and circular apertures.

4. A urine collection device of claim 1 wherein said collection bag has lines printed on said collection bag as measurement marks corresponding to the volume of urine collected.

5. A urine collection device of claim 1 wherein said stent retention means are at least a one paper tape attached to said collection bag at least one of said openings wherein said tape has an inwardly facing side coated with a pressure sensitive adhesive.

6. A urine collection device of claim 5 wherein said stent retention means has a pair of said paper tape at each of said openings.

7. A urine collection device of claim 1 wherein said stent retention means is at least one snap closure attached generally adjacent to at least one of said openings.

8. A urine collection device of claim 1 wherein said channel accommodates two stents.

9. A urine collection device of claim 1 wherein said collection bag has an aperture at a predetermined location in the bottom of said collection bag, further having a fitting, said fitting has a predetermined shape and size to receive one of a drainage tube and a cap.

10. A urine collection device of claim 1 further having collection bag securing means attached to the opposing lateral sides of said collection bag to secure said collection bag from free movement.

11. A urine collection device of claim 1 wherein said collection bag retention means are straps of predetermined lengths.

12. A urine collection device of claim 11 wherein said straps are one of cloth, paper and hook and loop fasteners.

13. A urine collection device of claim 1 wherein said first predetermined shape is one of slots and circles.
